(19) United States
(12) Patent Application Publication
(10) Pub. No.: US 2022/0339367 A1
(43) Pub. Date: Oct. 27, 2022

(54) INSULIN DELIVERY AND DATA COLLECTION SYSTEMS, INSULIN THERAPY MANAGEMENT SYSTEM, AND RELATED METHODS

(71) Applicant: Bigfoot Biomedical, Inc., Milpitas, CA (US)

(72) Inventors: Michael J. Wakim, Milpitas, CA (US); Stuart Helgeson, San Francisco, CA (US)

(21) Appl. No.: 17/660,244

(22) Filed: Apr. 22, 2022

Related U.S. Application Data
(60) Provisional application No. 63/201,365, filed on Apr. 26, 2021.

(51) Int. Cl. A61M 5/32

(52) U.S. Cl. CPC A61M 5/3202; A61M 2205/3393; A61M 2205/8237; A61M 2205/8243; A61M 2205/3553; A61M 2205/3592; A61M 2230/201; A61M 2205/18

(57) ABSTRACT

An insulin delivery and data collection system includes at least one insulin delivery device, a holding station, and control system. The holding station includes a body defining at least one cavity extending into the body from an upper surface of the body, the at least one cavity configured to receive the at least one insulin delivery device and at least one load cell disposed at a bottom of the at least one cavity. The control system includes at least one processor and at least one non-transitory computer-readable storage medium storing instructions thereon that, when executed by the at least one processor, cause the control system to receive weight data from the at least one load cell related to the at least one insulin delivery device.

### CROSS-REFERENCE TO RELATED APPLICATION

This application claims the benefit under 35 U.S.C. §119(e) of U.S. Provisional Patent Application Ser. No. 63/201,365, filed Apr. 26, 2021, the disclosure of which is hereby incorporated herein in its entirety by this reference.

### TECHNICAL FIELD

This disclosure relates generally to insulin delivery and data collection system, insulin therapy management systems, and methods of acquiring and analyzing insulin dosing data.

### BACKGROUND

Diabetes mellitus is a chronic metabolic disorder caused by the inability of a person's pancreas to produce sufficient amounts of the hormone insulin such that the person's metabolism is unable to provide for the proper absorption of sugar and starch. The inability to absorb those carbohydrates sometimes leads to hyperglycemia, i.e., the presence of an excessive amount of glucose within the blood plasma. Hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities.

Often, a permanent therapy is necessary to maintain a proper blood glucose level within normal limits. Maintaining a proper glucose level is achieved by regularly supplying insulin to a person with diabetes (PWD). Maintaining a proper blood glucose level creates a significant cognitive burden for a PWD and affects many aspects of the PWD's life. For example, the cognitive burden on a PWD may be attributed to, among other things, tracking meals and constant check-ins and minor course corrections of blood glucose levels. The adjustments of blood glucose levels by a PWD may include taking insulin, tracking insulin dosing and glucose, deciding how much insulin to take, how often to take it, where to inject the insulin, and how to time insulin doses in relation to meals and/or glucose fluctuations. The foregoing factors make up just a portion of the significant cognitive burden of a PWD.

The following example of a typical daily routine for a PWD further illustrates the significant cognitive burden of a PWD. In the morning, the first thoughts/actions by a PWD are often related to their glucose, such as, what is their blood glucose level? How was their blood glucose level overnight? And how are they currently feeling? Upon checking their blood glucose levels (e.g., using a blood glucose meter or monitor), a PWD may then consider what actions to take, such as adjusting their morning activities, changing when or what to eat for breakfast, or determining to take rapid-acting (RA) insulin and deciding where to injection the rapid-acting (RA) insulin. Before they even eat breakfast (or any meal), a PWD considers the amount of food and types of food they plan to eat, perhaps modifying their RA insulin dose based on the carbohydrate content of the food they choose to eat. Before they administer RA insulin, the PWD will try to remember when they took their last dose of insulin, what happened the last time they ate a particular meal and how they felt.

Before leaving the house, a PWD considers, among other things, whether they have enough supplies for glucose monitoring or insulin dosing. This may include batteries, charged devices, backup supplies, glucose testing supplies, and insulin supplies to treat for high blood glucose levels. Additionally, a PWD needs to consider any physical activities (e.g., walking kids to school, going to the gym, riding a bike) that will affect their glucose because exercise may cause their blood glucose to go lower than expected. Even before driving a vehicle, a PWD checks their glucose to determine if it is at a safe level for driving.

As lunchtime approaches, a PWD considers their glucose prior to eating lunch, such as what time they may expect to eat, what they expect to eat throughout the day. As such, a PWD tallies up the carbohydrates and adjusts insulin doses in their head. A PWD also considers what insulin doses were recently taken and whether those doses may still be working to lower blood glucose. This is all done in parallel with whatever they are doing in their busy day, and so the PWD often forgets or fails to fully consider all of the factors described above.

Throughout the day, a PWD often checks glucose levels, especially on days when their activities vary from a typical day. This constant thinking, checking, planning may be exhausting, especially when each check requires decisions, math, and possible behavior changes. Additionally, during the day, a PWD may check inventory on supplies, speak with a health care provider (HCP), refill prescriptions, and contact their health insurance to discuss their therapy and/or supplies.

In the evening, a PWD may have to take a daily insulin dose of long-acting (LA) insulin. Additionally, the PWD may determine if their glucose is holding steady before they fall asleep. If they use an infusion pump, they have to check if their insulin pump is low on insulin and whether they need to refill it before sleep. If they have a continuous glucose monitor, they have to check and see if it is working. Even then, based on what they ate for dinner, the nighttime insulin might not keep their glucose steady. Glucose levels in the night may interfere with sleep as well as add anxiety that could disrupt sleep.

Accordingly, managing diabetes requires significant attention to detail throughout the day. Even with careful planning and self-monitoring, a PWD may skip doses, double dose, or dose the wrong amount and/or type of insulin. Insufficient insulin may result in hyperglycemia, and too much insulin may result in hypoglycemia, which may result in clumsiness, trouble talking, confusion, loss of consciousness, seizures, or death.

It is often difficult for a caregiver to determine whether a PWD is complying with recommended insulin therapy regimes and whether the PWD is administering correct amounts of insulin per a given recommendation and/or injection because very little data is typically available in regard to how much insulin was actually administered within an insulin dose. Furthermore, conventional methods for measuring doses are limited to volume and/or optical methods, which are typically inaccurate and provide limited resolutions.

### BRIEF SUMMARY

Embodiments of the present disclosure include an insulin delivery data collection system that includes a holding station, and control system. The holding station may include a body defining at least one cavity extending into the body from an upper surface of the body, the at least one cavity configured to receive an at least one insulin delivery device and at least one load cell disposed at a bottom of the at least one cavity. The control system may include at least one processor and at least one non-transitory computer-readable storage medium having instructions stored thereon that, when executed by the at least one processor, at least partially enable the control system to receive weight data from the at least one load cell regarding the at least one insulin delivery device.

Some embodiments of the present disclosure include a method comprising receiving weight data related to an insulin delivery device and, based at least partially on the received weight data of the insulin delivery device, determining an amount of insulin administered over a selected period of time.

One or more embodiments of the present disclosure include an insulin delivery and data collection system. The insulin delivery and data collection system may include at least one insulin delivery device and a weighing system attached to a longitudinal end of the insulin delivery device. The weighing system may include at least one load cell disposed at a bottom of the at least one cavity and a control system. The control system may include at least one processor and at least one non-transitory computer-readable storage medium storing instructions thereon that, when executed by the at least one processor, cause the control system to receive weight data from the at least one load cell related to the at least one insulin delivery device.

### BRIEF DESCRIPTION OF THE DRAWINGS

Various embodiments will be described and explained with additional specificity and detail through the use of the accompanying drawings in which:

FIG. 1 shows a schematic representation of an environment within which an injection delivery and data collection system may operate in accordance with one or more embodiments;

FIG. 2A shows a perspective view of an injection delivery and data collection system according to one or more embodiments;

FIG. 2B shows a cross-sectional view of the injection delivery and data collection system of FIG. 2A;

FIG. 3 is a schematic representation of the holding station an injection delivery and data collection system according to one or more embodiments;

FIG. 4 shows a flow chart of a method of acquiring weight data of an insulin delivery device according one or more embodiments;

FIG. 5 shows a flow chart of a method of analyzing weight data according to one or more embodiments;

FIG. 6A shows a perspective view of an injection delivery and data collection system according to one or more embodiments;

FIG. 6B shows a cross-sectional view of the injection delivery and data collection system of FIG. 6A;

FIG. 7 illustrates a block diagram of an exemplary computing device in accordance with one or more embodiments;

FIG. 8 shows a side cross-sectional view of an insulin delivery and data collection system according to one or more additional embodiments of the disclosure;

FIG. 9A is a cross-sectional view of a load cell according to one or more embodiments of the disclosure;

FIG. 9B is a perspective view of the load cell of FIG. 9A;

FIG. 10A is a cross-sectional view of a load cell according to one or more embodiments of the disclosure;

FIG. 10B is a perspective view of the load cell of FIG. 10A; and

FIG. 11 is a side cross-sectional view of an insulin delivery and data collection system according to one or more additional embodiments of the disclosure.

### DETAILED DESCRIPTION

The illustrations presented herein are not actual views of any injection delivery and data collection system, or any component thereof, but are merely idealized representations, which are employed to describe the present invention.

As used herein, the singular forms following “a,” “an,” and “the” are intended to include the plural forms as well, unless the context clearly indicates otherwise.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

As used herein, any relational term, such as “first,” “second,” etc., is used for clarity and convenience in understanding the disclosure and accompanying drawings, and does not connote or depend on any specific preference or order, except where the context clearly indicates otherwise.

As used herein, the term “substantially” in reference to a given parameter, property, act, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measure of the given parameter, as well as variations resulting from manufacturing tolerances, etc.).

Embodiments include an insulin delivery and data collection system that captures weight data of an insulin delivery device (e.g., an injection pen) between administered insulin doses (e.g., dosing events). For example, embodiments include a charging and dose measuring station (e.g., a holding station) for weighing and charging one or more insulin delivery devices (e.g., injection pens). For instance, the holding station may provide one or more cavities (e.g., wells) that float independently relative to a base of the holding station and relative to one another. Additionally, the holding station may include a load cell within each of the one or more cavities, and the holding station may utilize the load cells to weigh the one or more insulin delivery devices when the one or more insulin delivery devices are inserted into the holding station for storage and/or charging. Furthermore, the holding station may transmit the captured weight data of the one or more insulin delivery devices to an insulin care management system on one or more servers (e.g., a cloud computing platform), and the insulin care management system may analyze the weight data along with any other available data (e.g., blood glucose data, time data, dosing data, etc.) to determine dosing amounts (e.g., changes in weight) per injection and/or over a given period of time. Based on the analysis, the insulin care management system may identify usage data and trends that can be utilized to optimize insulin therapy regimes for a given PWD. Additionally, the usage data may be utilized to determine a compliance level of a PWD in regard to a recommend insulin therapy regime.

The insulin delivery and data collection system may be designed to be attractive to use (e.g., to encourage use of the insulin delivery and data collection system), thereby increasing an amount of the data (e.g., weight data) that can be acquired. For instance, the insulin delivery and data collection system may be designed to utilize a minimal amount of space (e.g., desk space, counter space, etc.) and provide a convenient location to hold and store the insulin delivery device while charging the insulin delivery device.

Embodiments of the insulin delivery and data collection system of the present disclosure may be advantageous over conventional methods of measuring and/or estimating amounts of insulin doses or dosing over time. For example, in comparison to optical methods or mechanical methods (e.g., positional sensors for determining a position of a plunger within a syringe), which typically attempt to measure amounts of insulin doses or dosing over time via changes in volume (e g , milliliters), which can be relatively inaccurate and provide relatively low resolutions, the insulin delivery and data collection system of the present disclosure measure amounts of insulin dosing via changes in weight (e.g., mass), which may provide finer resolutions (e.g., resolutions of 1.0-5.0 mg, 5.0-10.0 mg, 10.0-15.0 mg, etc.). Therefore, the insulin delivery and data collection system may provide data (e.g., injection dose amounts, daily total usage data (e.g., total daily doses), etc.) with finer resolutions than conventional methods. Additionally, more accurate data may enable more accurate and more effective insulin therapy regimes to be determined and recommended for the PWD.

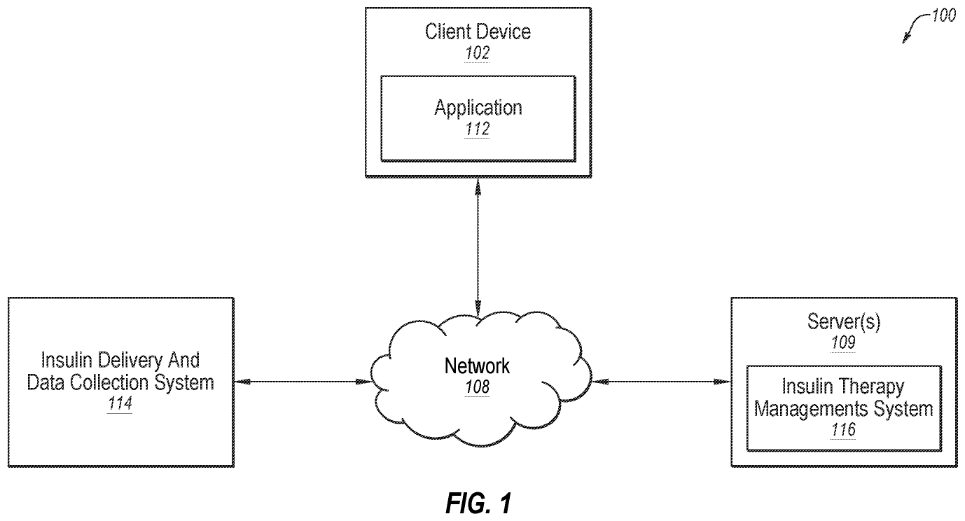

FIG. 1 illustrates a schematic diagram of an environment 100 (e.g., an overall system 100) in which an insulin delivery and data collection system (hereinafter “injection site determination system”) may operate according to one or more embodiments. As illustrated, the environment 100 includes an insulin delivery and data collection system 101, a client device 102, at least one server 109 including an insulin care management system 114, and a network 108. As is described in greater detail below, the insulin delivery and data collection system 101 may acquire measurements of a weight of an insulin delivery device over time (referred to hereinafter as “weight data”) and may provide the weight data to one or more of the client device or the insulin care management system 114. For example, the insulin delivery and data collection system 101 may acquire weight data of an insulin delivery device (described below) over a given period of time (e.g., a hour, 6 hours, 12 hours, 24 hours, etc.) and may transmit the acquired measurements to the insulin care management system 114 via the network. Responsive to receiving the measurements, the insulin care management system 114 may perform various analyses on and with the weight data to determine usage data and optimize recommended insulin therapy regimes.

In some embodiments, the client device 102 may include an application 112 (e.g., an insulin management application associated with the insulin care management system 114) for enabling a PWD to interact with the insulin care management system 114 and to manage its insulin therapy. As a non-limiting example, the application 112 may be directed to assisting a PWD in managing the insulin therapy of the PWD. In some instances, the application 112 may be a web application for managing insulin therapy of the PWD and for determining and recommending insulin therapy regimes. In some embodiments, the application 112 may be local to the client device 102. In other embodiments, the application 112 may be stored and/or at least partially operated via the insulin care management system 114. In some embodiments, the client device 102 may execute one or more applications (e.g., application 112) for performing the functions of the various embodiments and processes described herein.

In one or more embodiments, an application 112 may be a native application installed on the client device 102. For example, the application 112 may be a mobile application that installs and runs on a mobile device, such as a smart phone or a tablet. The application 112 may be specific to an operating system of the client device 102. Further, in some embodiments, the application 112 may be a client application that is associated with the insulin delivery and data collection system 101 and configured to enable interaction directly with the insulin delivery and data collection system 101 through the application 112.

As is described in greater detail below, the insulin delivery and data collection system 101 may include a delivery device and a measurement device (also referred to as a “holding station”). The measurement device may be utilized to acquire measurements (e.g., weight measurements) related to the delivery device. In some embodiments, the delivery device and the measurement device may be separate and distinct from each other. In other embodiments, the delivery device and the measurement device may form a single device.

The insulin delivery and data collection system 101, the client device 102, and the insulin care management system 114 may communicate via the network 108. In one or more embodiments, the network 108 may include a combination of cellular or mobile telecommunications networks, a public switched telephone network (PSTN), and/or the Internet or World Wide Web that facilitate the transmission of data between the insulin delivery and data collection system 101, the client device 102, and the insulin care management system 114. The network 108, however, may include various other types of networks that use various communication technologies and protocols, such as a wireless local network (WLAN), a wide area network (WAN), a metropolitan area network (MAN), other telecommunication networks, or a combination of two or more of the foregoing networks. In some embodiments, insulin delivery and data collection system 101, the client device 102, and the insulin care management system 114 may communicate via Bluetooth and Near-field communication in addition to or instead of the network 108.

Although FIG. 1 illustrates a particular arrangement of the insulin delivery and data collection system 101, the client device 102, the network 106, and the insulin care management system 114, various additional arrangements are possible. For example, the insulin delivery and data collection system 101 and/or the insulin care management system 114 may directly communicate with the client device 102, bypassing the network 108.

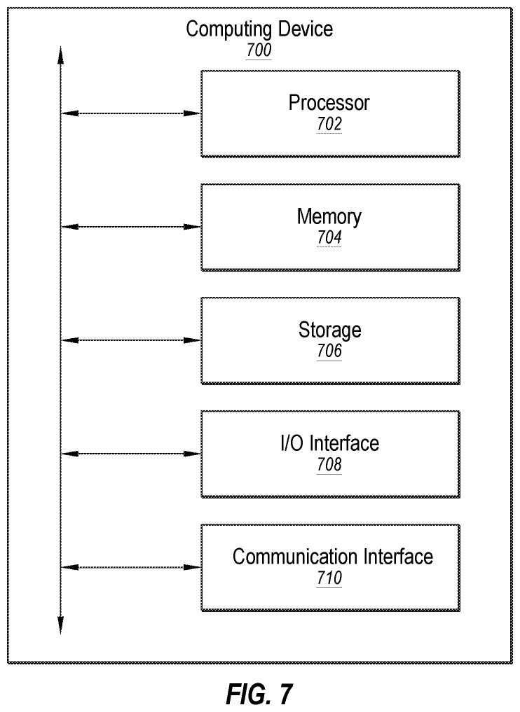

The client device 102 may be any one or more of various types of computing devices. For example, the client device 102 may include a mobile device such as a mobile telephone, a smartphone, a PDA, a tablet, or a laptop, or a non-mobile device such as a desktop or another type of computing device. Additional details with respect to the client device 102 are discussed below with respect to FIG. 7.

In some embodiments, the insulin care management system 114 may include one or more systems, servers, and/or other devices for managing insulin therapy regime for users (e.g., a person with diabetes (PWD)). Furthermore, the insulin care management system 114 may include and/or have access to one or more databases. For example, in some embodiments, an insulin care management system 114 may be implemented by a plurality of server devices that store, within the one or more databases, delivery device usage data, user preferences, glucose data, insulin therapy schedules/regimes, dosing schedules/regimes, dosing events, meal data, insulin-on-board data, etc. Furthermore, the insulin care management system 114 may include one or more analysis algorithms, machine-learning models, etc., for analyzing the data.

In some embodiments, the environment 100 may further include one or more external systems/resources that interface with one or more of the insulin delivery and data collection system 101, the client device 102, and the insulin care management system 114. For example, in some embodiments, the external systems/resources may include sources of information outside of the environment 100, external entities interacting with the environment 100, and/or other resources. In some embodiments, some or all of the functionality attributed herein to the external systems/resources may be provided by resources included in environment 100. The external systems/resources, in some embodiments, may include additional medical devices. The medical devices may include additional insulin delivery systems, including without limitation, insulin delivery devices (e.g., infusion pumps, injection pens, and inhalers), glucose sensors (e.g., CGMs and blood glucose meters), therapy managers (e.g., controllers for controlling open and closed-loop delivery of insulin or aspects of delivering insulin and recommendation systems for providing therapy recommendations to users and/or health providers), and combinations thereof. In some embodiments, the external systems/resources may include subject matter expert input data, clinical literature, conventional medication regimes, etc. The external systems/resources, in various embodiments, may include additional therapy management system(s). The therapy management systems may include a diabetes management system for monitoring blood glucose data and therapy data and managing therapy settings.

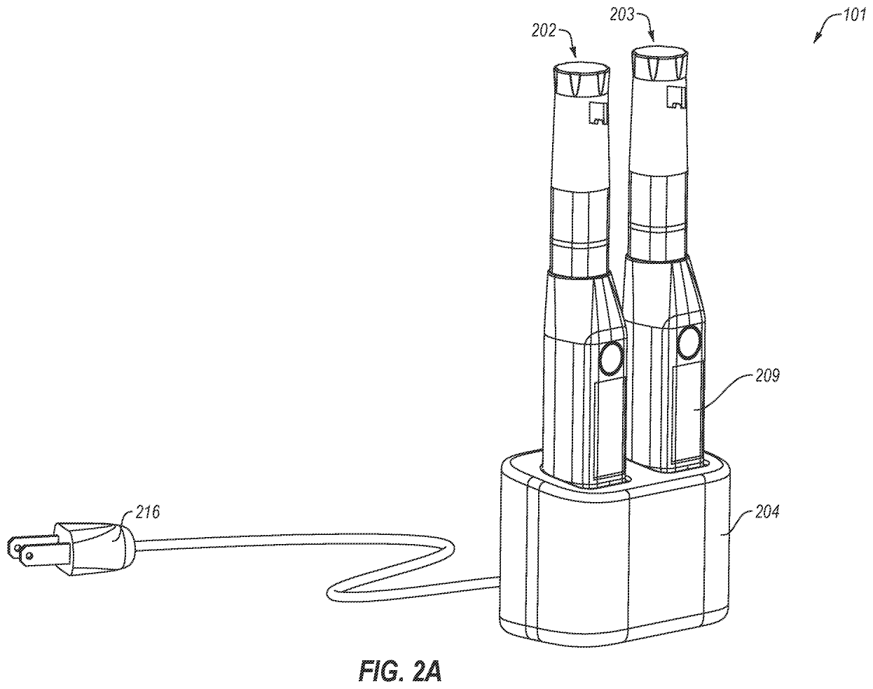

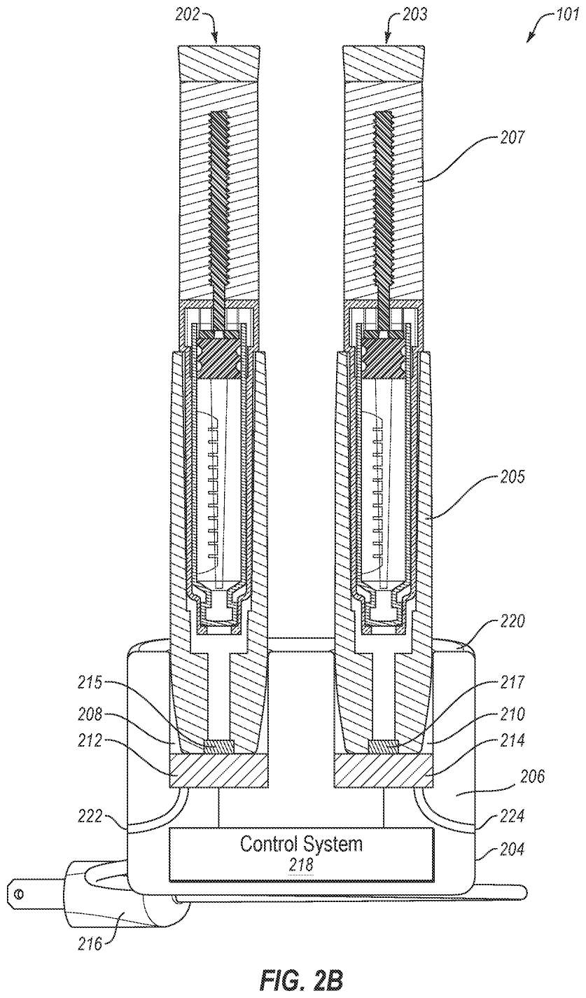

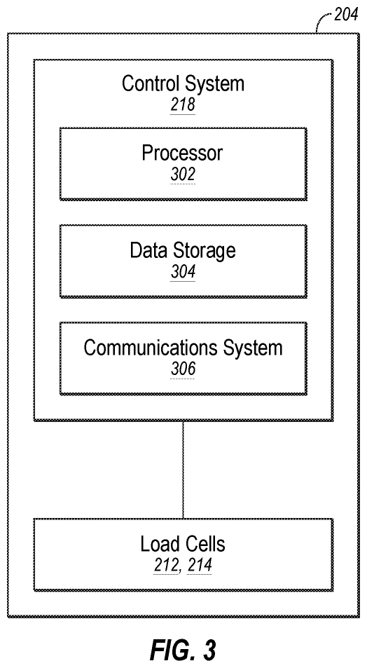

FIG. 2A is a perspective view of an insulin delivery and data collection system 101 according to one or more embodiments of the present disclosure. FIG. 2B is a side cross-sectional view of the insulin delivery and data collection system 101 of FIG. 2A. Referring to FIGS. 2A and 2B together, the insulin delivery and data collection system 101 may include one or more medication delivery devices 202, 203 and a holding station 204. In one or more embodiments, the holding station 204 may include a body 206 defining one or more cavities 208, 210 (e.g., wells), one or more load cells 212, 214, a power source 216, one or more charging elements 215, 217, and a control system 218. The control system 218 may be operably coupled to the one or more load cells 212, 214 and the power source 216 and is described in greater detail below in regard to FIG. 3.

In some embodiments, the one or more cavities 208, 210 (e.g., wells) may extend into the body 206 from an upper surface 220 of the body 206. Furthermore, each of the one or more cavities 208, 210 (e.g., wells) may be sized and shaped to receive at least a portion (e.g., a base portion) of a respective insulin delivery device of the one or more medication delivery devices 202, 203. For example, each of the one or more cavities 208, 210 may include an aperture extending downward into the body 206 of the holding station 204 and may be sized and shaped to hold a respective delivery device.

Additionally, each of the one or more cavities 208, 210 (e.g., wells) may include a respective load cell of the one or more load cells 212, 214 disposed at or proximate a bottom of the one or more cavities 208, 210 (e.g., wells). In particular, each of the one or more cavities 208, 210 may terminate at a lowermost boundary of the cavity at a respective load cell. For example, when the medication delivery device (e.g., medication delivery device 202) is disposed within a respective cavity, the medication delivery device 202 may rest on a respective load cell. Furthermore, as is discussed in greater detail below, when the medication delivery device 202 is disposed within the respective cavity, the control system 218 may receive data from the load cell to determine a weight and/or mass of the medication delivery device 202. Additionally, each of the one or more load cells 212, 214 may independently supported; in other words, each of the one or more load cells 212, 214 may float relative to the other such that each of the one or more medication delivery devices 202, 203 can be weighed independently.

Each of the one or more load cells 212, 214 may include a force transducer that converts force (e.g., compression) into an electrical signal that can be measured to determine a weight on the respective load cell. In one or more embodiments, each of the one or more load cells 212, 214 may include a strain gauge load cell. For instance, each of the one or more load cells 212, 214 may include a body (e.g., a metal body) with one or more strain gauges secured thereto. The strain gauges may include any conventional strain gauge. As noted above, the one or more load cells 212, 214 may be communicatively coupled to the control system 218, and the one or more load cells 212, 214 may provide any generated electrical signals to the control system 218.

In one or more embodiments, the power source 216 may include a cable and plug for connecting to conventional power outlets. In additional embodiments, the power source 216 may include one or more batteries (e.g., rechargeable batteries). Furthermore, the power source 216 may include any conventional power source. Additionally, the power source 216 may be operably coupled to the control system 218 of the holding station 204, and during use, the power source 216 may provide power to one or more elements of the holding station 204.

In one or more embodiments, the one or more medication delivery devices 202, 203 may include one or more injection pens 207 (e.g., an insulin injection pens). In some embodiments, the one or more medication delivery devices 202, 203 may further include one or more pen caps 205 (e.g., dose-capture caps). In some embodiments, the one or more medication delivery devices 202, 203 may include one or more of a quick acting insulin (QAI) pen or a long acting insulin (LAI) pen. In other embodiments, the one or more medication delivery devices 202, 203 may include any known insulin pump. In embodiments where the one or more medication delivery devices 202, 203 include one or more or more pen caps 205, the one or more pen caps 205 may be in wireless communication with one or more of the client device 102, the application 112 of the client device 102, the holding station 204, a glucose monitor, and/or the insulin care management system 114. In some embodiments, the injection pen 207 may include a display screen for displaying dosing information, medication (e.g., insulin) information, and/or recommendations. The injection pen 207 may further include one or more inputs (e.g., dials, buttons, and/or touch screen regions) for a user to set a dosage to be delivered. In some embodiments, the injection pen 207 and/or the pen caps 205 may itself include dose-capture technology and/or may be in wireless communication with other components of the environment 100.

In some embodiments, the pen cap 205 of a respective medication delivery device 202, 203 may include a display screen 209 for displaying one or more of an estimated glucose value (EVG), units for the EVG, a trend indicator for the EVG, a recommended dosage, an identification of the type of medication (e.g., insulin), a recommended site injection, a time and amount of a previous dosage, and/or an insulin on board value to remind a user about their most recent dosage. The pen cap 205 may include buttons for inputting meals information, inputting insulin dose information, responding to recommendations, etc. The pen cap 205 may include one or more indicator lights, which may light up to indicate that it is transferring data, light up to indicate that the user's attention is needed, and/or light up to indicate whether a dose capture functionality is or is not working. In some embodiments, the display screen 209 of the pen cap 205 may include a touch screen, which may include the one or more buttons. As a non-limiting example, the one or more medication delivery devices 202, 203 may include any of the insulin delivery pens described in U.S. Pat. No. 10,426,896, issued Oct. 1, 2019, to Desborough et al., the contents and disclosure of which is incorporated herein in its entirety by this reference.

In one or more embodiments, the one or more charging elements 215, 217 may be within or proximate the one or more cavities 208, 210 and may be utilized to charge one or more power sources (e.g., a rechargeable batteries) of the one or more medication delivery devices 202, 203. For example, in some embodiments, the one or more charging elements 215, 217 may include one or more contact elements that are sized and shaped to contact one or more correlating contact elements of the one or more medication delivery devices 202, 203 and provide power through the resulting connection. In additional embodiments, the one or more charging elements 215, 217 may include one or more inductive elements configured to wirelessly transfer power (e.g., transfer power through inductive coupling) to correlating receiving elements of the one or more medication delivery devices 202, 203. Moreover, the one or more charging elements 215, 217 may include any other conventional elements for transferring power.

In some embodiments, the one or more charging elements 215, 217 may include a USB plug. For example, the one or more charging elements 215, 217 may include one or more of a type-A or type B plug (2.0 or 3.0), a mini-USB connector, a micro-USB connector, or a USB-C connector. Additionally, the one or more medication delivery devices 202, 203 may include correlating receptacles for receiving the one or more charging elements 215, 217. In additional embodiments, the one or more charging elements 215, 217 may include any conventional plug or connector utilized for transferring power.

The one or more charging elements 215, 217 may be operably coupled to the control system 218, and the control system 218 may control charging processes of the powers sources of the one or more medication delivery devices 202, 203. Furthermore, during use, the control system 218 may monitor a charge level of the power sources (e.g., a rechargeable batteries) of the one or more medication delivery devices 202, 203. Additionally, when the control system 218 determines that the power sources of the one or more medication delivery devices 202, 203 are at full charge levels, the control system 218 may terminate charging processes. As a result, the control system 218 may prevent damaging the power sources of the one or more medication delivery devices 202, 203 by preventing overcharging the power sources.

In one or more embodiments, the one or more charging elements 215, 217 may also be utilized to transfer data from the holding station 204 (e.g., the control system 218 of the holding station 204) to the one or more medication delivery devices 202, 203 or from the one or more medication delivery devices 202, 203 to the holding station 204 (e.g., the control system 218 of the holding station 204).

In some embodiments, the holding station 204 may further include one or more drain holes 222, 224 in fluid communication with a bottom of the one or more cavities 208, 210 to permit fluids to freely drain from the one or more cavities 208, 210 and to reduce a likelihood that fluids will damage components of the holding station 204.

In further embodiments, the holding station 204 may include one or more of a night light, a clock display, or one or more power outlets (e.g., power outlets for charging a phone) to encourage use of the holding station 204.

FIG. 3 shows a schematic diagram of the holding station 204 according to one or more embodiments of the present disclosure. Referring to FIGS. 1-3 together, in some embodiments, the holding station 204 may include the control system 300 including a processor 302, data storage 304 (e.g., memory), and a communications system 306. The communication system 306 may enable wireless communication between the holding station 204 and an access point (e.g., a router), and ultimately, the insulin care management system 114 (e.g., one or more cloud computing platforms) via the network 106 and Internet. The communication system 306 may further enable wireless communication between the holding station 204 and one or more medication delivery devices 202, 203 or the client device 102.

The communication system 306 can include hardware, software, or both. In any event, the communication system 306 can provide one or more interfaces for communication (such as, for example, packet-based communication) between the holding station 204, the medication delivery devices 202, 203, and the client devices 102 or networks. As an example and not by way of limitation, the communication system 306 may include a network interface controller (NIC) or network adapter for communicating with an Ethernet or other wire-based network or a wireless NIC (WNIC) or wireless adapter for communicating with a wireless network, such as aWI-FI.

Additionally or alternatively, the communication system 306 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the communication system 306 may facilitate communications with a wireless PAN (WPAN) (such as, for example, a BLUETOOTH®WPAN), a WI-FI network, a WI-MAX network, a cellular telephone network (such as, for example, a Global System for Mobile Communications (GSM) network), or other suitable wireless network or a combination thereof.

Additionally, the communication system 306 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

As mentioned above, in some embodiments, the communications system 306 may further enable wireless communication between the holding station 204 and the client device 102 (e.g., a mobile phone) and/or the one or more medication delivery devices 202, 203. In some instances, the communications system 306 may further include a near field communications (NFC) chip. For example, the communications system 306 may include a Bluetooth Low Energy (BLE) chip. In some embodiments, the communications system 306 may include one or more of an optical communication device, an infrared communication device, a wireless communication device (such as an antenna), and/or chipset (such as a Bluetooth device (e.g., Bluetooth Low Energy, Classic Bluetooth, etc.), a Near-field communication (NFC) device, an 802.6 device (e.g., Metropolitan Area Network (MAN), a Zigbee device, etc.), a WiFi device, a WiMax device, cellular communication facilities, etc.), and/or the like. In these and other cases, the communications system 306 may exchange data with the network 108 and/or any other device or system described in the present disclosure.

Referring to still to FIGS. 1-3 together, as is described in greater detail herein, during use, a user (e.g., a PWD) may place a medication delivery device (e.g., insulin delivery devices 202, 203 (referred to hereinafter collectively as “medication delivery device 202” for ease of explanation)) into a cavity 208 of the holding station 204, and the holding station 204 (e.g., the control system 300 of the holding station 204) may measure a weight (e.g., capture a weight) of the medication delivery device 202 and may communicate the weight data to the insulin care management system 114. Additionally, the holding station 204 may capture the weight data over a period of time (e.g., may capture multiple instances of weight data over a period of time including multiple insertions into and removals out of the holding station 204). Furthermore, by capturing weight data over time in conjunction with time data (e.g., captured times at which the medication delivery device 202 is removed from and replaced within the holding station 204), the insulin care management system 114 may determine (e.g., identify) usage data correlating to the PWD.

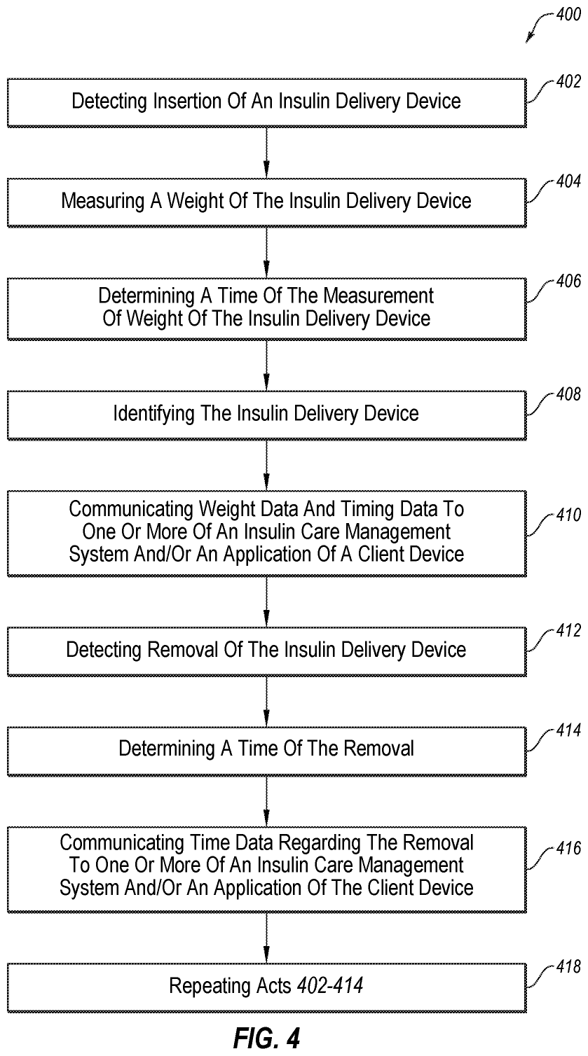

FIG. 4 shows a flowchart of a method 400 of acquiring weight data of the medication delivery device (e.g., medication delivery device 202). The method 400 may include detecting insertion of a medication delivery device 202 into a cavity (e.g., cavity 208) of the holding station (e.g., holding station 204) (also referred to herein as an “insertion event”), as shown in act 402 of FIG. 4. For example, the control system 218 of the holding station 204 may detect insertion of the medication delivery device 202 into the cavity 208. In some embodiments, detecting insertion of the medication delivery device 202 into the cavity 208 of the holding station 204 may include detecting creation of a connection between a charging element (e.g., charging element 215) of the holding station 204 and a correlating contact of the medication delivery device 202. In additional embodiments, detecting insertion of the medication delivery device 202 into the cavity 208 of the holding station 204 may include detecting a force on a load cell (e.g., load cell 212). In further embodiments, detecting insertion of the medication delivery device 202 into the cavity 208 of the holding station 204 may include detecting insertion of the medication delivery device 202 via a sensor (e.g., an optical sensor or other conventional sensor).

Responsive to detecting insertion of the medication delivery device 202 into the cavity 208 of the holding station 204, the method 400 may include measuring a weight of the medication delivery device 202, as shown in act 404 of FIG. 4. For example, the control system 218 of the holding station 204 may measure a weight of the medication delivery device 202 via the load cell (e.g., load cell 212) within the cavity 208 of the holding station 204. For instance, the control system 218 may receive a voltage generated by the load cell 212 in response to an applied force or pressure (e.g., the weight of the medication delivery device 202) and may determine weight data of the medication delivery device 202 based on the received voltage. The method 400 may further include acquiring charge level data of the medication delivery device 202 (e.g., an amount of battery life remaining in the medication delivery device 202). In some embodiments, the control system 218 may acquire the charge level data via any conventional methods.

Additionally, the method 400 may include determining a time of the measurement of weight of the medication delivery device 202, as shown in act 406 of FIG. 4. In some embodiments, the method 400 may include receiving time data from one or more of the insulin care management system 114 and/or the client device 102. For instance, the control system 218 of the holding station 204 may receive the time data from one or more of the insulin care management system 114 and/or the client device 102. In additional embodiments, the control system 218 may include a time keeping element (e.g., a clock) and may acquire the time data from the time keeping element. Additionally, the method 400 may include associating the time data with the determined weight data. For instance, the control system 218 may store a data package within a database associating the time data with the determined weight data.

Furthermore, the method 400 may include identifying the medication delivery device 202, as shown in act 408 of FIG. 4. In other words, the method 400 may include determining which medication delivery device has been inserted into the holding station 204. For example, in some embodiments, the method 400 may include identifying the medication delivery device 202 based on a comparison of current weight data of the medication delivery device 202 and previous weight data of the medication delivery device. For instance, the method 400 may include determining a correlation between the current weight data of the medication delivery device 202 and the previous weight data. In some embodiments, the method 400 may include identifying the medication delivery device 202 based on a comparison of a current charge level of a power source (e.g., a rechargeable battery) of the medication delivery device 202 and a previously known charge level of device medication delivery device. In further embodiments, the method 400 may include identifying the medication delivery device 202 based on communication with the medication delivery device 202. For instance, the method 400 may include receiving an identification token from the medication delivery device 202 through one or more of wireless communication or through the charging elements 215, 217. Additionally, the method 400 may include identifying the medication delivery device 202 via any conventional method of identifying objects. In some embodiments, the control system 218 of the holding station 204 may identify the medication delivery device 202. In other embodiments, the insulin care management system 114 and/or the application 112 of the client device 102 may identify the medication delivery device 202 responsive to receiving weight data via one or more of the manners described above. In some embodiments, the holding station 204 may associate the weight data and the timing data with an identity of the medication delivery device 202 (e.g., an identification number of the medication delivery device 202).

The method 400 may further include communicating the weight data of the medication delivery device 202 to one or more of the insulin care management system 114 and/or the application 112 of the client device 102, as shown in act 410 of FIG. 4. For example, the control system 218 of the holding station 204 may transmit a data package include the weight data of the medication delivery device 202 to one or more of the insulin care management system 114 and/or the application 112 of the client device 102. Furthermore, in embodiments where the control system 218 includes a time keeping element (e.g., a clock) and the time data is acquired from the time keeping element, the control system 218 of the holding station 204 may transmit a data package including the time data to one or more of the insulin care management system 114 and/or the application 112 of the client device 102. Moreover, the control system 218 may include transmitting the identification data of the medication delivery device 202 to the one or more of the insulin care management system 114 and/or the application 112 of the client device 102. Additionally, in some embodiments, responsive to receiving the data package regarding the measurement of the weight of the medication delivery device 202, the insulin care management system 114 or the application 112 of the client device 102 may transmit the data package to the other of the insulin care management system 114 and/or the application 112 of the client device 102.

Additionally, the method 400 may include detecting removal of the medication delivery device 202 from the cavity 208 of the holding station 204 (also referred to herein as a “removal event”), as shown in act 412 of FIG. 4. For example, the control system 218 of the holding station 204 may detect removal of the medication delivery device 202 from the cavity 208 of the holding station 204. In some embodiments, detecting removal of the medication delivery device 202 from the cavity 208 of the holding station 204 may include detecting a breaking of a connection between the charging element 215 of the holding station 204 and a correlating element of the medication delivery device 202 (e.g., detecting breaking of a connection between contacts). In additional embodiments, detecting removal of the medication delivery device 202 from the cavity 208 of the holding station 204 may include detecting removal or a sudden absence of a force on the load cell 212. In further embodiments, detecting removal of the medication delivery device 202 from the cavity 208 of the holding station 204 may include detecting removal of the medication delivery device 202 via a sensor (e.g., an optical sensor or other conventional sensor).

Additionally, the method 400 may include determining a time of the removal of the medication delivery device 202 from the holding station 204, as shown in act 414 of FIG. 4. In some embodiments, the method 400 may include receiving time data from one or more of the insulin care management system 114 or the client device 102. For instance, the control system 218 of the holding station 204 may receive the time data from one or more of the insulin care management system 114 or the client device 102. In additional embodiments, as noted above, the control system 218 may include a time keeping element (e.g., a clock) and may acquire the time data from the time keeping element. Additionally, the method 400 may include associating the time data (e.g., time of removal) with a data package representing the medication delivery device 202.

Furthermore, the method 400 may include communicating the time data regarding the removal of the medication delivery device 202 to one or more of the insulin care management system 114 and/or the application 112 of the client device 102, as shown in act 416 of FIG. 4.

Moreover, the method 400 may include repeating acts 402-414 any number of times as a PWD utilizes and charges the medication delivery device 202. For example, the holding station 204 may measure the weight of the medication delivery device 202 whenever the medication delivery device 202 is disposed within the holding station 204 and may communicate the weight data along with time data to one or more of the insulin care management system 114 or the client device 102. Furthermore, as is described in greater detail below, the holding station 204, the application 112 of the client device 102, and/or the insulin care management system 114 may analyze the weight data and the timing data and may determine usage data (e.g., usage patterns, doses over time, individual doses, user tendencies, user compliance with insulin therapy regimes, etc.). Furthermore, the usage data may be utilized to adjust, optimize, and/or determine insulin therapy regimes (e.g., insulin dose recommendations).

Referring still to method 400 and FIG. 4, the method 400 may further include storing the weight data, charge level data, and/or the time data. For example, in some embodiments, the control system 218 may store the weight data, the charge level data, and/or the time data locally in the holding station 204 within a database. In additional embodiments, the control system 218 may communicate the weight data, the charge level data, and/or the time data to one or more of the application 112 of the client device 102 or the insulin care management system 114, and the application 112 of the client device 102 and/or the insulin care management system 114 may store the weight data, the charge level data, and/or the time data within the client device 102 or the cloud, respectively.

Additionally, in some embodiments, one or more of the holding station 204 (e.g., the control system 218), the application 112 of the client device 102, or the insulin care management system 114 may receive data from the medication delivery device 202. For instance, the one or more of the holding station 204 (e.g., the control system 218), the application 112 of the client device 102, or the insulin care management system 114 may receive dosing data (e.g., capping and uncapping data) from the medication delivery device 202, and the dosing data may be transmitted to and from one or more the holding station 204 (e.g., the control system 218), the application 112 of the client device 102, or the insulin care management system 114 via any of the manners described above.

Referring still to FIG. 4, in some embodiments, the holding station 204 may transmit data to the application 112 of the client device 102, which may, in turn, transmit the data to the insulin care management system 114. In additional embodiments, the holding station 204 may transmit the data directly to the insulin care management system 114.

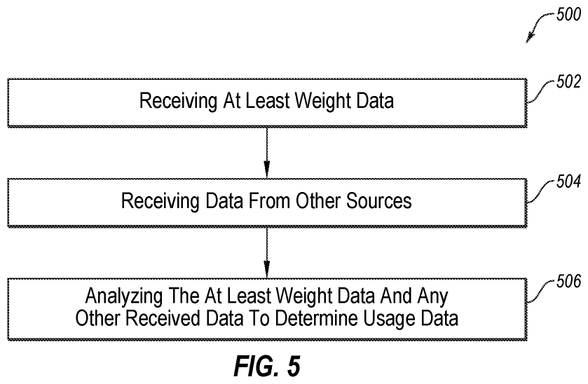

FIG. 5 depicts a flowchart of a method 500 of determining usage data from at least weight data of the medication delivery device 202. The method 500 is described herein as being performed by the insulin care management system 114; however, the disclosure is not so limited. Rather, it is understood that one or more acts of method 500 may be performed (e.g., accomplished) by the control system 218 of the holding station 204 and/or the application 112 of the client device 102 and by sharing data between the holding station 204, the application 112 of the client device 102, and the insulin care management system 114.

The method 500 may include receiving at least weight data from the holding station 204, as shown in act 502 of FIG. 5. In some embodiments, the insulin care management system 114 may receive the at least weight data from the holding station 204 directly via the network 106 or from the holding station 204 through the client device 102 (e.g., application 112 of client device 102) via the network 106. In some embodiments, receiving the at least weight data may include receiving the weight data in addition to at least one of charge level data, identification data, and timing data from the holding station 204. Additionally, receiving the at least weight data may include receiving multiple instances of weight data (e.g., data reflecting multiple weigh-ins) over a period of time (e.g., one day, two days, one week, two weeks, one month, two months, one year, or more).

Additionally, the method 500 may optionally include receiving data from other sources, as shown in act 504 of FIG. 5. For example, the method 500 may include receiving blood glucose data from a blood glucose monitor, capping and/or uncapping data from the medication delivery device 202, or user preferences from the client device 102. The insulin care management system 114 may receive the foregoing data directly or through the holding station 204 and/or the client device 202.

Responsive to receiving the at least weight data from the holding station 204, the method 500 may include analyzing the at least weight data and any other received data to identify usage data, as shown in act 506 of FIG. 5. In some embodiments, identifying usage data may include identify usage trends of a PWD.

For example, identifying usage data may include identifying when, during a day, a PWD is most likely to use the medication delivery device. For instance, based at least partially on the timing data (e.g., insertions and removal times of the insulin delivery device) and the charge level data (e.g., changes in charge levels between insertions), the insulin care management system 114 may determine when a PWD is most likely to administer insulin. Furthermore, the insulin care management system 114 may determine whether the PWD administers insulin in response to a recommendation, alert, notification, a meals, etc.

As another non-limitation example, identifying usage data may include identifying an amount of medication used over a period of time (e.g., a day). For example, based on the weight data, the insulin care management system 114 may determine an amount of medication (e.g., insulin) utilized between insertion events in weight (e.g., mass), and based on the amount of medication utilized between insertion events, the insulin care management system 114 may determine an amount of medication utilized for a given injection, an hour period, a 6-hour period, a 12-hour period, a day, a week, a month, etc. For instance, the insulin care management system 114 may determine an amount of medication delivered between insertion events based on differences in weight measurements at those insertion events.

As would be expected, a user may not reinsert the medication delivery device 202 into the holding station 204 after every use (e.g., injection). Therefore, in some embodiments, the insulin care management system 114 may utilize the weight data in combination with one or more of charge levels data, uncapping or capping data, and blood glucose data to estimate an amount of medication utilized for individual doses. For example, the charge level data may generally indicate a number of injections performed between insertion events when considered with the timing data (e.g., times of insertion events) and a known general decay rate of the power source (e.g., battery) of the medication delivery device 202. Furthermore, uncapping and/or capping data (e.g., data related to the uncapping and/or capping of the pen cap 205 from the medication delivery device 202) from the medication delivery device 202 may further inform a number of injections performed between insertion events based on uncapping and/or capping events.

Additionally, identifying usage data may include determining how the PWD uses insulin cartridges within the medication delivery device 202. For example, the insulin care management system 114 may determine whether the PWD tends to use substantially all of a cartridge prior to replacing the cartridge or whether the PWD tends to be more cautious and replaces the cartridge earlier than is necessarily needed. For instance, based on the weight data, the insulin care management system 114 may know a remaining amount of insulin within a cartridge and, as a result, may know an anticipated number of remaining doses within the cartridge. Furthermore, based on a step-wise jump (e.g., step-wise increase) in weight of the medication delivery device 202 relative to a previously measured weight (e.g., an indication that the cartridge has been replaces), the insulin care management system 114 may determine whether the cartridge was replaced prematurely based on the previously measured weight, and an estimated number of administered injections since the previously measured weight.

Furthermore, identifying usage data may include determining whether a PWD is following instructions from a caregiver. For example, based on the received weight data, the charge level data, and/or the received timing data, the insulin care management system 114 may determine whether the PWD is administering insulin dosing according to instructions (e.g., recommendations) from an care giver (e.g., according to a recommended insulin therapy regime).

Moreover, identifying usage data may include determining usage trends of the medication delivery device 202. For instance, in embodiments including at least two medication delivery devices (e.g., medication delivery devices 202, 203), identifying usage data may include determining usage trends for each of the medication delivery devices 202, 203 and/or usage trends of the medication delivery devices 202, 203 relative to each other. For instance, based on the weight data over time, the insulin care management system 114 may determine that a PWD prefers to use one of the medication delivery devices 202, 203 more than the other. For example, one of the medication delivery devices 202, 203 may include a LAI pen and the other of the medication delivery devices 202, 203 may include a QAI pen. Furthermore, the insulin care management system 114 may determine that a PWD prefers to use one of a LAI pen or a QAI pen more than the other or prefers to use a first LAI pen more than a second LAI pen, etc.

In some embodiments, the insulin care management system 114 may analyze at least the weight data and any other received data to determine usage data via one or more machine learning techniques and/or deep learning techniques. For example, the insulin care management system 114 may utilize one or more machine learning techniques to iteratively model and predict usage data where data may be missing from the weight data and any other received data. Furthermore, the insulin care management system 114 may utilize one or more machine learning techniques to model usage trends and to identify usage patterns. In some embodiments, the machine learning and/or deep learning techniques may include one or more of regression models (e.g., a set of statistical processes for estimating the relationships among variables), classification models, and/or phenomena models. Additionally, the machine-learning techniques and/or deep learning techniques may include a quadratic regression analysis, a logistic regression analysis, a support vector machine, a Gaussian process regression, ensemble models, or any other regression analysis. Furthermore, in yet further embodiments, the machine-learning techniques and/or deep learning techniques may include decision tree learning, regression trees, boosted trees, gradient boosted tree, multilayer perceptron, one-vs-rest, Naïve Bayes, k-nearest neighbor, association rule learning, a neural network, deep learning, pattern recognition, or any other type of machine-learning.

Furthermore, based on the determined usage data, embodiments of the present disclosure include determining and recommending insulin therapy regimes based on the determined usage data. Additionally, embodiments of the present disclosure include providing recommendation to adjust insulin therapy regimes of a PWD based at least partially on the determined usage data.

Referring to FIGS. 1-5 together, the insulin delivery and data collection system 101 may be advantageous over conventional methods of measuring and/or estimating amounts of insulin dosing. For example, in comparison to optical methods or mechanical methods (e.g., positional sensors (e.g., positional sensors for determining a position of a plunger within a syringe)), which typically attempt to measure amounts of insulin dosing via changes in volume (e.g., milliliters), which can be relatively inaccurate and provide low resolutions, the insulin delivery and data collection system 101 of the present disclosure measure amounts of insulin dosing via changes in weight (e.g., mass), which may provide finer resolutions (e.g., resolutions of 1.0-5.0 mg, 5.0-10.0 mg, 10.0-15.0 mg, etc.). Therefore, the insulin delivery and data collection system 101 may provide data (e.g., daily total usage data (e.g., total daily doses)) with finer resolutions than conventional methods. Additionally, more accurate data may enable more accurate and more effective insulin therapy regimes to be determined and recommended for the PWD.

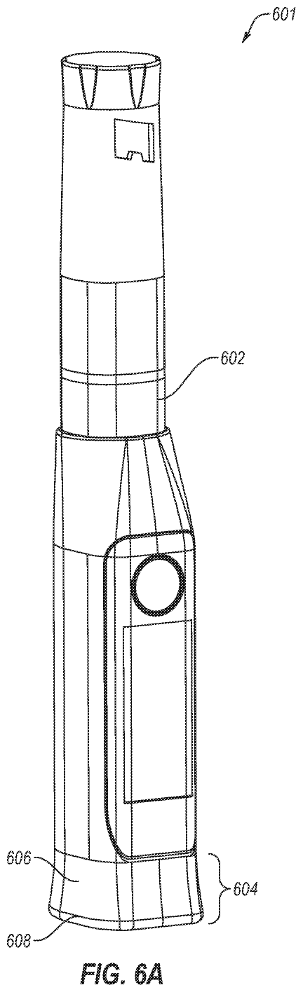

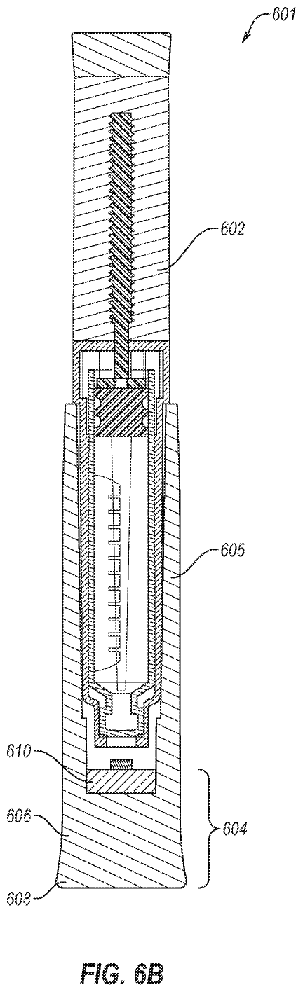

FIG. 6A is a perspective view of an insulin delivery and data collection system 601 according to one or more embodiments of the present disclosure. FIG. 6B is a side cross-sectional view of the insulin delivery and data collection system 601 of FIG. 6A. In some embodiments, the insulin delivery and data collection system 601 may include a single injection pen and pen cap combination. The injection pen and pen cap combination may include both insulin medication delivery device 602 (e.g., injection pen) and a pen cap 605 having weighing system 604. For example, the medication delivery device 602 may include any of the insulin delivery devices described above in regard FIGS. 1-5. Additionally, the pen cap 605 having the weighing system 604 may be coupled to (e.g., capped onto) a longitudinal end (e.g., a needle end) of the medication delivery device 602 and may be sized and shaped to hold the medication delivery device 602 in a vertical position. For instance, the pen cap 605 having the weighing system 604 may include a base portion 606 having a flared bottom portion 608 for resting against a horizontal surface upon which the insulin delivery and data collection system 601 may be disposed (e.g., stood up). Additionally, the weighing system 604 may include a load cell 610 disposed within the pen cap 605 and which may be utilized to capture weight date of the medication delivery device 602. The pen cap 605 and the weighing system 604 may include any of the components of the control system 218 of the holding station 204 described above. Furthermore, the insulin delivery and data collection system 601 may communicate with and transfer to the insulin c are management system 114 and/or the client device 102 via any of the manners described above in regard to FIGS. 1-5.

In some embodiments, the pen cap 605 having the weighing system 604 may include a distinct unit that can be attached to an off-the-shelf medication delivery device 602.

FIG. 7 is a block diagram of an exemplary computing device 700 that may be utilized as a client device (e.g., client device 102) and/or an injection site determination system (e.g., insulin delivery and data collection system 101) that may be configured to perform one or more of the processes described above. One will appreciate that one or more computing devices may implement the computing device 700. The computing device 700 may comprise a processor 702, a memory 704, a storage device 706, an I/O interface 708, and a communication interface 710, which may be communicatively coupled by way of a communication infrastructure 712. While an exemplary computing device is shown in FIG. 7, the components illustrated in FIG. 7 are not intended to be limiting. Additional or alternative components may be used in other embodiments. Furthermore, in certain embodiments, the computing device 700 may include fewer components than those shown in FIG. 7. Components of the computing device 700 shown in FIG. 7 will now be described in additional detail.

In one or more embodiments, the processor 702 includes hardware for executing instructions, such as those making up a computer program. As an example and not by way of limitation, to execute instructions, the processor 702 may retrieve (or fetch) the instructions from an internal register, an internal cache, the memory 704, or the storage device 706, and decode and execute them. In one or more embodiments, the processor 702 may include one or more internal caches for data, instructions, or addresses. As an example and not by way of limitation, the processor 702 may include one or more instruction caches, one or more data caches, and one or more translation lookaside buffers (TLBs). Instructions in the instruction caches may be copies of instructions in the memory 704 or the storage device 706.

The memory 704 may be used for storing data, metadata, and programs for execution by the processor(s). The memory 704 may include one or more of volatile and non-volatile memories, such as Random Access Memory (“RAM”), Read-Only Memory (“ROM”), a solid state disk (“SSD”), Flash memory, Phase Change Memory (“PCM”), or other types of data storage. The memory 704 may be internal or distributed memory.

The storage device 706 includes storage for storing data or instructions. As an example and not by way of limitation, storage device 706 may comprise a non-transitory storage medium described above. The storage device 706 may include a hard disk drive (HDD), a floppy disk drive, flash memory, an optical disc, a magneto-optical disc, magnetic tape, or a Universal Serial Bus (USB) drive or a combination of two or more of these. The storage device 706 may include removable or non-removable (or fixed) media, where appropriate. The storage device 706 may be internal or external to the computing device 700. In one or more embodiments, the storage device 706 is non-volatile, solid-state memory. In other embodiments, the storage device 706 includes read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these.

The I/O interface 708 allows a user to provide input to, receive output from, and otherwise transfer data to and receive data from computing device 700. The I/O interface 708 may include a mouse, a keypad or a keyboard, a touch screen, a camera, an optical scanner, network interface, modem, other known I/O devices or a combination of such I/O interfaces. The I/O interface 708 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 708 is configured to provide graphical data to a display for presentation to a user. The graphical data may be representative of one or more graphical user interfaces and/or any other graphical content as may serve a particular implementation.

The communication interface 710 may include hardware, software, or both. In any event, the communication interface 710 may provide one or more interfaces for communication (such as, for example, packet-based communication) between the computing device 700 and one or more other computing devices or networks. As an example and not by way of limitation, the communication interface 710 may include a network interface controller (NIC) or network adapter for communicating with an Ethernet or other wire-based network or a wireless NIC (WNIC) or wireless adapter for communicating with a wireless network, such as a WI-FI.

Additionally or alternatively, the communication interface 710 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the communication interface 710 may facilitate communications with a wireless PAN (WPAN) (such as, for example, a BLUETOOTH®WPAN), a WI-FI network, a WI-MAX network, a cellular telephone network (such as, for example, a Global System for Mobile Communications (GSM) network), or other suitable wireless network or a combination thereof.

Additionally, the communication interface 710 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

The communication infrastructure 712 may include hardware, software, or both that couples components of the computing device 700 to each other. As an example and not by way of limitation, the communication infrastructure 712 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

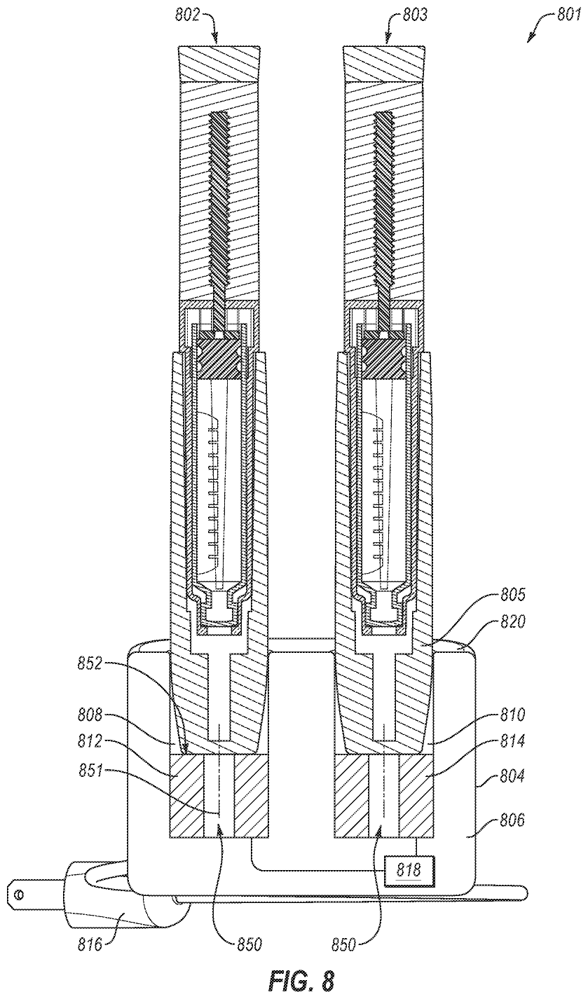

FIG. 8 is a side cross-sectional view of the insulin delivery and data collection system 801 according to one or more additional embodiments of the disclosure. Similar to the insulin delivery and data collection system 101 described above, the insulin delivery and data collection system 801 may include one or more medication delivery devices 802, 803 and a holding station 804. In one or more embodiments, the holding station 804 may include a body 806 defining one or more cavities 808, 810 (e.g., wells), one or more load cells 812, 814, a power source 816, one or more charging elements (not shown for clarity), and a control system 818. The control system 818 may be operably coupled to the one or more load cells 812, 814 and the power source 816 and may operate in any of the manners described above in in regard to FIG. 3.

The structure of the holding station 804 may be substantially the same as the structure of holding station 204 described above. For instance, each of the one or more cavities 808, 810 (e.g., wells) may include a respective load cell of the one or more load cells 812, 814 disposed at or proximate a bottom of the one or more cavities 808, 810 (e.g., wells). However, each of the load cells 812, 814 may define a central cavity 850 extending from a top surface (i.e., resting surface 852) of the respective load cell axially into the respective load cell. In some embodiments, the central cavity 850 may be substantially centered on a center vertical axis 851 of the respective load cell. In view of the foregoing, in some embodiments, each of the load cells 812, 814 may have general hollow-cylinder shape. Furthermore, each of the load cells 812, 814 may provide a support profile having at least one raised outer radial portion and a recessed center radial portion. In some embodiments, the at least one raised outer radial portion may include a plurality of raised outer radial portions. In one or more embodiments, the plurality of raised outer radial portions may be oriented in a circle shape (e.g., donut shape) and may form a segmented circle. Each of the load cells 812, 814 may define a general circular-shaped (i.e., donut-shaped) resting surface 852 for supporting an insulin delivery device (e.g., one of more insulin delivery devices 802, 803). In some embodiments, each of the load cells 812, 814 may include a plurality of load cells.

When medication delivery device (e.g., medication delivery device 802) is disposed within a respective cavity, the medication delivery device 802 may rest on the resting surface 852 a respective load cell, and the control system 818 may receive data from the load cell to determine a weight and/or mass of the medication delivery device 202. Moreover, because each of the load cells 812, 814 has a generally circular-shaped resting surface 852 and defines a respective central cavity 850, the medication delivery device 802 may be disposed with or without a respective pen cap 805 (e.g., dose-capture cap) and with or without a needle. For instance, when the medication delivery device 802 is disposed without a pen cap 805 and with a needle, the needle may be inserted into a respective central cavity 850, and the medication delivery device 802 may rest on the circular-shaped resting surface 852 while suspending the needle.

Each of the load cells 812, 814 may include a force transducer that converts force (e.g., compression) into an electrical signal that can be measured to determine a weight on the respective load cell. For example, each of the load cells 812, 814 may include any of the load cells described above. As noted above, the one or more load cells 812, 814 may be communicatively coupled to the control system 818, and the one or more load cells 812, 814 may provide any generated electrical signals to the control system 818. Moreover, in some embodiments, based on the received electrical signals, the control system 818 may determine where on a respective resting surface 852 a given load cell is experiencing a load. Furthermore, based on a determination on where the load cell is experiencing a load, the control system 818 may determine whether or not a respective insulin delivery device has been inserted with a pen cap attached. For example, a smaller surface area of the respective resting surface 852 experiencing a load may indicate that a pen cap has not been inserted, and a larger surface area of the respective resting surface 852 experiencing a load may indicate that a pen cap has been inserted. Furthermore, the presence or absence of the pen cap may be utilized in any of the calculations described above.

In alternative embodiments, the medication delivery devices 802 may include one or more additional sensors (e.g., optical sensors, inductors, proximity sensors, near field communication sensors, switches, contacts, etc.) for determining whether the pen cap 805 has been inserted with the medication delivery device 802.

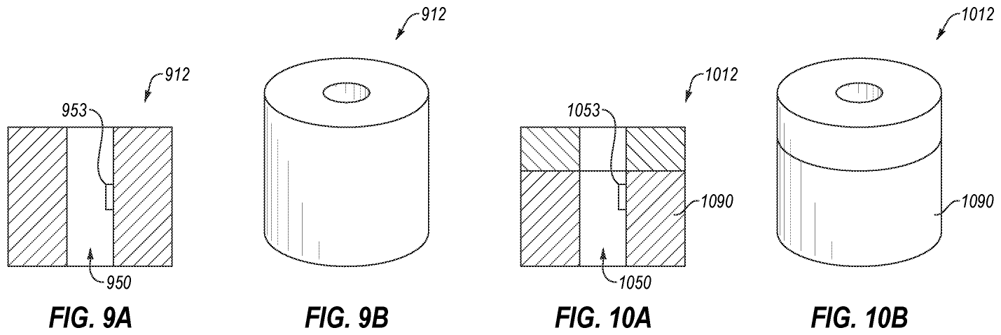

FIG. 9A is a cross-sectional view of a load cell 912 according to one or more embodiments of the disclosure. FIG. 9B is a perspective view of the load cell 912 of FIG. 9A. Referring to FIGS. 9A-9B together, the load cell 912 may further include a needle sensor 953. In some embodiments, the needle sensor 953 may be disposed within a given central cavity 950 of the load cell 912. In one or more embodiments, the needle sensor 953 may be disposed at a based on the given central cavity 950 of the load cell 912. Furthermore, the needle sensor 953 may be operably coupled to a control system 818 (FIG. 8) and may be configured to sense the presence or absence of a needle and/or sense whether a needle is attached to a given injection pen 802, 803 when the injection pen 802, 803 is inserted into the holding station 804. The needle sensor 953 may include one or more of an optical sensor, a magnetic sensor, a switch, a proximity sensor, or any other sensor for detecting the presence of an object. In some embodiments, the control system 818 may receive data from the needle sensor 953, and the control system 818 may utilize the data in any of the analyses described herein (e.g., the analyses described above in regard to FIG. 5). Determining whether a needle is attached to a given injection pen 802, 803 when the injection pen 802, 803 is inserted into the holding station 804 may yield more accurate results in determining usage data.

FIG. 10A is a cross-sectional view of a load cell 1012 according to one or more embodiments of the disclosure. FIG. 10B is a perspective view of the load cell 1012 of FIG. 10A. As shown in FIGS. 10A and 10B, in some embodiments, the load cell 1012 may be disposed on top of a support body 1090, and the support body 1090. The support body 1090 may also have a center cavity 1050 to permit a needle to be received therein such that the load cell 1012 may fully support a given injection pen 802, 803.

Referring to FIGS. 8-10B together, in some embodiments, the insulin delivery and data collection system 801 may further include a motion sensor that may be utilized by the control system 818 to determine when a given injection pen 802, 803 is motionless and when to perform a weight measurement.

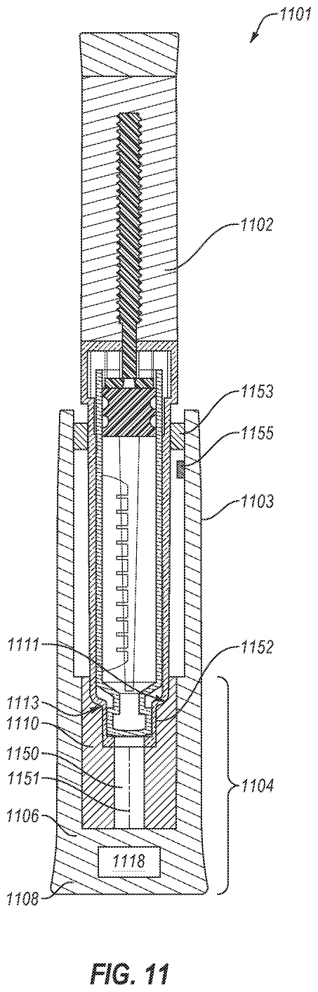

FIG. 11 is a side cross-sectional view of the insulin delivery and data collection system 1101 according to one or more embodiments of the disclosure. Similar to the embodiments described above in regard to FIGS. 6A and 6B, the insulin delivery and data collection system 1101 may include an injection pen and pen cap combination, which includes both an insulin delivery device 1102 and the pen cap 1103 includes a weighing system 1104. For example, the insulin delivery device 1102 may include any of the insulin delivery devices described above in regard FIGS. 1-5. Additionally, the pen cap 1103 including a weighing system 1104 may be engageable to a bottom end (e.g., needle end) of the insulin delivery device 1102. The pen cap 1103 may be sized and shaped to hold the insulin delivery device 1102 in a vertical orientation. For instance, the weighing system 1104 may include a base portion 1106 having a flared bottom portion 1108 for resting against a horizontal surface upon which the insulin delivery and data collection system 1101 may be disposed (e.g., stood up).

The pen cap 1103 and the weighing system 1104 may include a load cell 1110 disposed within the pen cap 1103 at an end of the pen cap 103 opposite an opening into which the insulin delivery device 1102 may be inserted. The load cell 1110 may have an upper surface 1111 (e.g., resting surface 1152) that is substantially congruent with a longitudinal end surface 1113 of the insulin delivery device 1102. In other words, a shape of the upper surface 1111 of the load cell 1110 may substantially matches a shape of the longitudinal end surface 1113 of the insulin delivery device 1102. The load cell 1110 may further define a central cavity 1150 extending from the upper surface 1111 into the load cell 1110. In some embodiments, the central cavity 1150 may be substantially centered on a center vertical axis 1151 of the load cell 1110. In view of the foregoing, in some embodiments, the load cell 1110 may have general hollow-cylinder shape.

The pen cap 1103 may further include one or more electromechanical clasping mechanisms 1153 for grasping the insulin delivery device 1102 (e.g., injection pen). The electromechanical clasping mechanisms 1153 may be operated between engaged orientations and disengaged orientations via the controller 1118. Furthermore, the controller 1118 may operate and receive information from the load cell 1110 via any of the manners described above. The pen cap 1103 may further include a sensor 1155 for determining an orientation of the pen cap 1103 and/or the insulin delivery device 1102. For example, the controller 1118 may utilize information from the sensor 1155 to determine the pen cap 1103 and/or the insulin delivery device 1102 are in a vertical orientation.

In some embodiment, a wall defining an inner cavity of the pen cap 1103 into which the insulin delivery device 1102 (e.g., injection pen) may be received (e.g., inserted) may define an inner boundary (e.g., diameter) that is larger than an outer boundary (e.g., diameter) of the insulin delivery device 1102 (e.g., injection pen) such that when the insulin delivery device 1102 is disposed within the pen cap 1103, the insulin delivery device 1102 is spaced apart from a wall of the pen cap 1103. As a result, when the insulin delivery device 1102 is properly disposed within and engaged with the pen cap 1103, the insulin delivery device 1102 may contact only the load cell 1110 and the electromechanical clasping mechanisms 1153, when engaged.

The load cell 1110 may include a force transducer that converts force (e.g., compression) into an electrical signal that can be measured to determine a weight being experienced on the load cell 1110. For example, the load cell 1110 may include any of the load cells described above. As noted above, the load cell 1110 may be communicatively coupled to the controller 1118, and the load cell 1110 may provide any generated electrical signals to the controller 1118. When the insulin delivery device 1102) is disposed within the pen cap 1103, the insulin delivery device 1102 may rest on the resting surface 1152 of the load cell 1110, and the controller 1118 may receive data from the load cell to determine a weight and/or mass of the insulin delivery device 1102. Moreover, because the load cell 1110 has a general cylindrical shape and defines a respective central cavity 1150, the insulin delivery device 1102 may be disposed with or without a needle. For instance, when the insulin delivery device 1102 is disposed with a needle, the needle may be inserted into the central cavity 1150, and the insulin delivery device 1102 may rest on the circular-shaped resting surface 1152 while suspending the needle.

During operation and during a weigh operation, when the insulin delivery device 1102 is disposed within the pen cap 1103, and the pen cap 1103 is placed on a horizontal surface, the controller 1118 may determine that the pen cap 1103 and/or the insulin delivery device 1102 are in a vertical orientation. For example, the controller 1118 may determine that the pen cap 1103 and/or the insulin delivery device 1102 are in a vertical orientation based on information received from the sensor 1155. Responsive to determining that the pen cap 1103 and/or the insulin delivery device 1102 are in a vertical orientation, the controller 1118 may disengage the electromechanical clasping mechanisms 1153 from the insulin delivery device 1102. Furthermore, because the upper surface 1111 (e.g., resting surface 1152) of the load cell 1110 is substantially congruent with the longitudinal end surface 1113 of the insulin delivery device 1102, the load cell 1110 may hold the insulin delivery device 1102 in a vertical orientation. Moreover, because the electromechanical clasping mechanisms 1153 are disengaged from the insulin delivery device 1102 and because the wall defining the inner cavity of the pen cap 1103 is spaced apart from the insulin delivery device 1102, an entirety of the weight of the insulin delivery device 1102 is supported by the load cell 1110.

Furthermore, controller 1118 may communicate with and transfer data to the insulin care management system 114 (FIG. 1) and/or the client device 102 (FIG. 1) via any of the manners described above in regard to FIGS. 1-5.

Referring still to FIG. 11, in some embodiments, the controller 1118 may be configured to provide a notification when the controller 1118 is determining an orientation of the pen cap 1103 and/or the insulin delivery device 1102. For instance, the controller 1118 may be configured to provide an indication on a display screen of the pen cap 1103 that the controller 1118 is determining an orientation of the pen cap 1103 and/or the insulin delivery device 1102. In additional embodiments, the controller 1118 may be configured to provide an audio indication that the controller 1118 is determining an orientation of the pen cap 1103 and/or the insulin delivery device 1102. In further embodiments, the controller 1118 may be configured to provide an indication via the client device 102 (FIG. 1) that the controller 1118 is determining an orientation of the pen cap 1103 and/or the insulin delivery device 1102.

Additionally, the controller 1118 may be configured to provide a notification when the controller 1118 is performing a weight measurement of the insulin delivery device 1102. For instance, the controller 1118 may be configured to provide an indication on a display screen of the pen cap 1103 that the controller 1118 is performing a weight measurement. As a non-limiting example, the indication may read “DO NOT TOUCH: PERFORMING WEIGHT MEASUREMENT.” In additional embodiments, the controller 1118 may be configured to provide an audio indication that the controller 1118 is performing a weight measurement of the insulin delivery device 1102. In further embodiments, the controller 1118 may be configured to provide an indication via the client device 102 (FIG. 1) that the controller 1118 is performing a weight measurement of the insulin delivery device 1102.

The embodiments of the disclosure described above and illustrated in the accompanying drawing figures do not limit the scope of the invention, since these embodiments are merely examples of embodiments of the invention, which is defined by the appended claims and their legal equivalents. Any equivalent embodiments are intended to be within the scope of this invention. Indeed, various modifications of the present disclosure, in addition to those shown and described herein, such as alternative useful combinations of the content features described, may become apparent to those skilled in the art from the description. Such modifications and embodiments are also intended to fall within the scope of the appended claims and legal equivalents.

We claim:

1. An insulin delivery data collection system, comprising:
a holding station comprising:
a body defining at least one cavity extending into the body from an upper surface of the body, the at least one cavity configured to receive an at least one insulin delivery device; and
at least one load cell disposed at a bottom of the at least one cavity; and
a control system comprising:
at least one processor; and
at least one non-transitory computer-readable storage medium having instructions stored thereon that, when executed by the at least one processor, at least partially enable the control system to receive weight data from the at least one load cell regarding the at least one insulin delivery device.

2. The insulin delivery data collection system of claim 1, wherein body comprises:
at least two cavities: and
at least two load cells, each load cell being disposed within a respective cavity of the at least two cavities.

3. The insulin delivery data collection system of claim 2, wherein the at least two load cells float freely relative to each other and operate independently relative to each other.

4. The insulin delivery data collection system of claim 1, further comprising at least one charging element disposed within the at least one cavity and configured to interact with a correlating charging element of the at least one insulin delivery device.

5. The insulin delivery data collection system of claim 4, further comprising instructions that, when executed by the at least one processor, cause the control system to:
monitor a charge level of a power source of the at least one insulin delivery device; and
acquire a charge level of the power source of the at least one insulin delivery device responsive to insertion of the at least one insulin delivery device into the at least one cavity.

6. The insulin delivery data collection system of claim 4, wherein the at least one charging element comprises at least one USB plug.

7. The insulin delivery data collection system of claim 4, wherein the at least one charging element comprises at least one inductive charging plate or coil.

8. The insulin delivery data collection system of claim 4, further comprising instructions that, when executed by the at least one processor, cause the control system to transfer data to or receive data from the at least one insulin delivery device through the at least one charging element.

9. The insulin delivery data collection system of claim 1, wherein the at least one load cell comprises a strain gauge load cell.

10. The insulin delivery data collection system of claim 1, wherein the at least one cavity is configured to receive an injection pen.

11. The insulin delivery data collection system of claim 4, further comprising instructions that, when executed by the at least one processor, cause the control system to wireles sly transmit the weight data to one or more of a cloud computing platform or a client device.

12. A method, comprising:
receiving weight data related to an insulin delivery device; and
based at least partially on the received weight data of the insulin delivery device, determining an amount of insulin administered over a selected period of time.

13. The method of claim 12, wherein determining an amount of insulin administered over a selected period of time comprises determining an amount of insulin administered during a single injection.

14. The method of claim 12, wherein determining an amount of insulin administered over a selected period of time comprises determining an amount of insulin administered during for a period of an hour, six hours, 12 hours, a day, or a week.

15. The method of claim 12, wherein receiving weight data related to an insulin delivery device comprises receiving the weight data responsive to the insulin delivery device being inserted into a holding device.

16. The method of claim 12, comprising receiving timing data related to when the weight data was captured.

17. The method of claim 16, further comprising receiving one or more of blood glucose data related to a person with diabetes (PWD), capping and uncapping data of the insulin delivery device, or user preferences data related to insulin therapy.

18. The method of claim 17, further comprising:
analyzing one or more of the weight data, the timing data, the blood glucose data, the capping and uncapping data, or the user preferences data;
based on the analysis, determining usage data related to the insulin delivery device; and
based at least partially on the determined usage data, changing a parameter of a recommend insulin therapy regime.

19. The method of claim 18, wherein determining usage data comprises determining when the PWD is most likely to use the insulin delivery device.

20. The method of claim 18, wherein determining usage data comprises determining whether the PWD is likely to administer insulin responsive to one or more of a recommendation, alert, notification, or meal.

21. The method of claim 18, wherein determining usage data comprises determining preferences of a first insulin delivery device over a second insulin delivery device.

22. The method of claim 18, wherein analyzing one or more of the weight data, the timing data, the blood glucose data, the capping and uncapping data, or the user preferences data comprises utilizing one or more machine learning model to perform the analysis.

23. An insulin delivery and data collection system, comprising:
at least one insulin delivery device;
a weighing system attached to a longitudinal end of the insulin delivery device, the weighing system comprising:
at least one load cell disposed at a bottom of at least one cavity; and
a control system comprising:
at least one processor; and
at least one non-transitory computer-readable storage medium storing instructions thereon that, when executed by the at least one processor, cause the control system to receive weight data from the at least one load cell related to the at least one insulin delivery device.
